(19) United States
(12) Patent Application Publication
(10) Pub. No.: US 2018/0110495 A1
(43) Pub. Date: Apr. 26, 2018

(54) AUTOMATED WIRELESS DETECTOR POWER-UP FOR IMAGE ACQUISITION

(71) Applicant: Carestream Health, Inc., Rochester, NY (US)

(72) Inventor: Scott T. MacLaughlin, Pittsford, NY (US)

(21) Appl. No.: 15/298,338

(22) Filed: Oct. 20, 2016

(51) Int. Cl. A61B 6/00

(52) U.S. Cl. CPC A61B 6/56; H04W 88/08; A61B 6/4233

(57) ABSTRACT

A radiographic imaging system includes a radiographic energy source, a digital radiographic detector with a wireless transceiver, and a wireless router to transmit and to receive electromagnetic signals from a pair of antennae that are spaced apart. A host processor in signal communication with the wireless router is programmed to determine motion and location of persons proximate the system according to the received signals, and to output an activation signal to energize the detector in preparation for image acquisition.

### FIELD OF THE INVENTION

The invention relates generally to the field of medical imaging and more particularly relates to a digital x-ray imaging detector and methods for providing automated power-up capability.

### BACKGROUND

Digital radiography (DR) imaging detectors convert incident x-ray radiation energy to pixelated digital image data using a scintillator material that converts the x-ray energy to visible light for detection by an array of photodetectors. DR detectors typically have a housing that supports and protects the scintillator material and its accompanying photodetector array and also contains various other types of circuitry for providing power, data processing, control, and data communication for the detector.

Wireless DR detectors are configured to acquire and process image data from an x-ray exposure of a patient or other subject and to communicate the digital image data to a host computer using a wireless router or similar transceiver circuits. Wireless transmission eliminates the need for interconnecting cables between the DR detector and computer host, and simplifies requirements for mounting and use of the DR detector in a retrofit assembly for use in older stationary facilities using film cassette based x-ray apparatuses for image acquisition.

One difficulty with wireless DR operation in older retrofit systems relates to controlling the power-up function for remote devices. The DR detector must be properly initialized immediately before an exposure in order to be ready for imaging and to provide accurate and useful radiographic image data. The detector should not be run continuously, since this would generate a considerable amount of unneeded data and consume battery power. Using beam-detection logic to sense incident radiation levels that indicate an exposure has begun are unsatisfactory because they can add to patient x-ray dose and consume power with the detector in a waiting state. Controlling DR detector power-up can be further complicated by site-specific differences between equipment configurations from different vendors; this can be particularly complex where a DR detector has been added to a site as a retrofit. Related practical limitations, as well as possible regulatory complications, can make it difficult or unfeasible to adapt existing equipment to provide a timely wireless power-up signal, or other activation signals, to the DR detector.

Timing considerations for controlling DR detector power up have both workflow and image quality implications. It is advantageous to energize the DR detector to a ready state just prior to exposure, without requiring a lengthy waiting time. And because patient motion can cause undesirable blurring of the image, limiting the wait interval just before exposure is advantageous for image sharpness.

It would be beneficial to be able to power up and initialize the DR detector immediately before it is needed to acquire radiographic image data from an x-ray exposure, to provide suitable power for transforming the received radiographic energy to digital data, to wirelessly transmit the generated digital image data upon completion of the exposure cycle, and to shut power down, all without x-ray technician intervention, in order to reduce detector power consumption and eliminate unnecessary generation and transmission of x-rays and detector image data between patient exams.

### SUMMARY

An aspect of this application is to advance the art of medical digital radiography and to address, in whole or in part, at least the foregoing and other deficiencies of the related art. It is another aspect of this application to provide in whole or in part, at least the advantages described herein. For example, certain exemplary embodiments of the application address the need to provide automated power-up for DR detector image acquisition.

According to one aspect of the disclosure, there is provided a radiographic imaging apparatus comprising a radiographic energy source, a digital radiographic detector including a wireless transceiver, a wireless router disposed to transmit and to receive a wireless signal from a first antenna and from a second antenna that is spaced apart from the first antenna, a host processor in signal communication with the wireless router and programmed with instructions to sense motion proximate the first and second antennae according to the received wireless signals, and to provide an output signal that energizes the detector in preparation for image acquisition.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other.

FIG. 1 is an exploded, perspective view showing components of a DR detector, as packaged within a housing.

FIG. 2 is an exploded, perspective view showing components of a DR detector according to an alternate packaging embodiment.

FIG. 3 is a schematic diagram showing an x-ray site that provides wireless communication with a DR detector.

FIG. 4A is a schematic diagram that shows tracking of patient motion.

FIG. 4B is a schematic diagram that shows tracking of practitioner motion.

FIG. 5 is a top view of an x-ray site showing detection of both practitioner and patient movement into position for imaging.

FIG. 6 is a logic flow diagram for power-up and imaging processing that can be executed using the x-ray site apparatus shown in FIG. 5.

FIG. 7 is a logic flow diagram showing a power-down sequence for the wireless DR detector.

FIG. 8 is a logic flow diagram that shows a sequence that can be used for setup and “training” of the wireless DR detector power-up function.

### DESCRIPTION OF EXEMPLARY EMBODIMENTS

The following is a description of exemplary embodiments, reference being made to the drawings in which the same reference numerals identify the same elements of structure in each of the several figures.

Where they are used in the present disclosure, the terms “first”, “second”, and so on, do not necessarily denote any ordinal, sequential, or priority relation, but are simply used to more clearly distinguish one element or set of elements from another, unless specified otherwise.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating digital data with each other via signals that travel over a wireless or wired signal path. The signals may be communication signals, power signals, data signals, energy signals, or a combination thereof. The signal paths may include physical, electrical, magnetic, electromagnetic, optical, wired, and/or wireless connections between a first device and/or component and a second device and/or component. The signal paths may also include additional devices and/or components between the first device and/or component and the second device and/or component.

In the context of the present disclosure, the terms “operator”, “user”, and “viewer” are used equivalently and refer to the technician, radiographer, or other practitioner who operates an x-ray system or facility used for exposure and imaging of a patient.

Reference is hereby made to U.S. Pat. No. 8,189,124 to Tsai et al. entitled “Digital photo frame with a function of automatically power off”; U.S. Pat. No. 7,409,564 to Kump et al. entitled “Digital radiography detector with thermal and power management”; U.S. Pat. No. 6,650,322 to Dai et al. entitled “Computer screen power management through detection of user presence”; U.S. Pat. No. 8,237,696 to Chung et al. entitled “Intelligent digital photo frame”, all of which are incorporated by reference herein in their entirety; and Hambling, David, “Seeing Through Walls With a Wireless Router” Popular Science online posting dated Aug. 1, 2012 at http: web site address www.popsci.com under /technology/article/2012-07/seeing-through-walls-wireless-router.

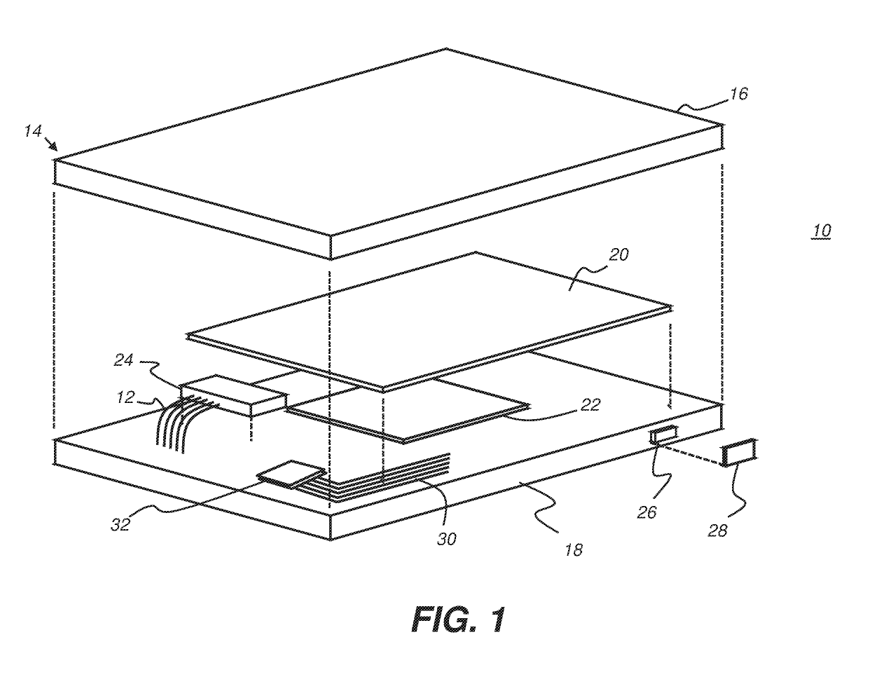

The exploded view of FIG. 1 shows, in simplified form, some of the electrically active internal components of a DR detector 10 that are protected within an enclosure or housing 14 formed using multiple parts, including top and bottom covers 16 and 18. A detector array 20 includes a recording medium, a scintillator layer that outputs light energy when energized under x-ray exposure, and electromagnetic radiation sensitive elements (imaging pixels) disposed in a two-dimensional array for capturing image signals from received radiation. A circuit board 22 provides supporting control electronics components for image data acquisition and wireless transmission to an external host system. Circuit board 22 includes electric circuits to initiate a start of exposure and to terminate the exposure. A battery 24 provides power, acting as the voltage source for detector 10 operations. A port 26 extending through bottom cover 18 is provided to allow electrical connection for receiving and transmitting data, and/or receiving power such as from a voltage supply. The port may have an optional cover plate or sealing cap 28, which may be a rubber seal or other liquid-proofing material. In addition to the illustrated components, a number of interconnecting cables, supporting fasteners, cushioning materials, connectors, and other elements may be used for packaging and protecting the DR detector circuitry. An optional antenna 30 and transmitter circuitry 32 for wireless communication may be provided, with antenna 30 extending within the housing 14 along an interior surface of the housing. Top and bottom housing covers 16 and 18 may be fastened together along a common mating surface. One or more cables 12, such as multi-wire flexible cables, may also be included within housing 14 for interconnection between components.

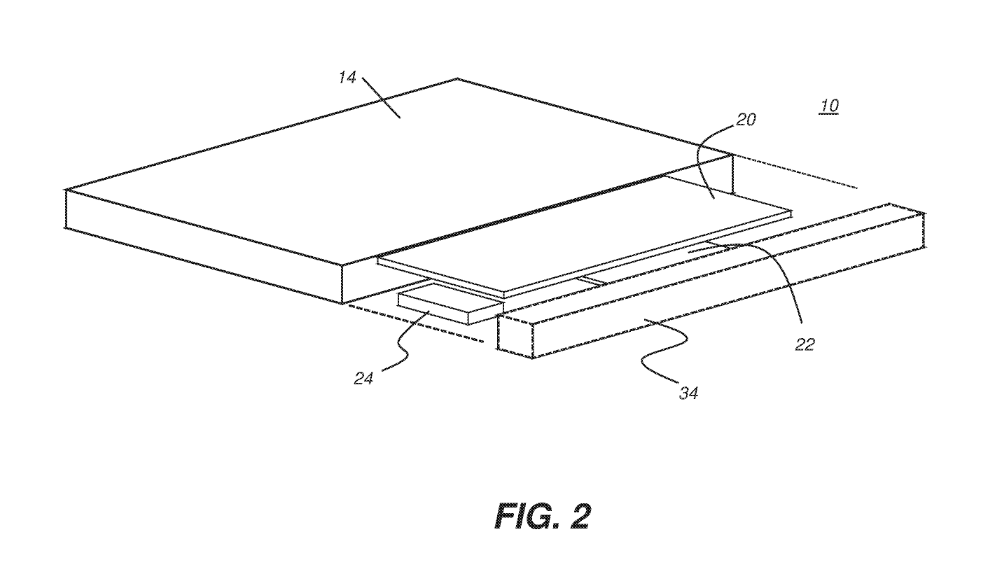

The exploded view of FIG. 2 shows an alternate embodiment of DR detector 10, in which detector array 20, circuit board 22, and battery 24, along with interconnection and other support components, slide into an encased cavity in an enclosure or housing 14 through an open end thereof. A lid 34 may be fastened to housing 14 to provide a protective seal.

The rechargeable battery 24 for the wireless DR detector is typically a Lithium-ion battery (LIB) battery pack, often used for portable electronics devices. Alternately, a storage capacitor, such as a supercapacitor or ultracapacitor, can be used for providing portable device power.

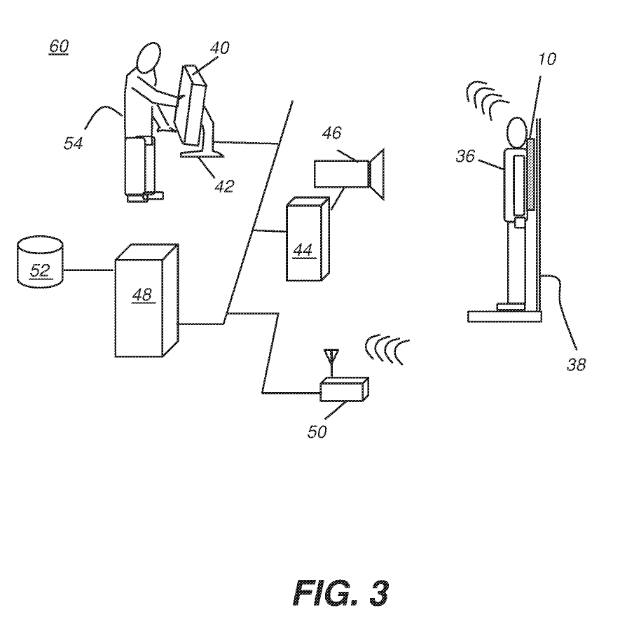

The schematic diagram of FIG. 3 shows an x-ray site 60, such as a medical imaging facility or an x-ray imaging room, for example, that is configured for wireless communication with DR detector 10. A patient 36 may be positioned for an x-ray imaging against a wall stand bucky 38 or other apparatus that holds wireless DR detector 10 in a fixed position during imaging. At a control console 40 that includes a display 42, a practitioner 54 controls a generator 44 that excites x-ray emission from an x-ray energy source 46. X-ray source 46 and generator 44 can include a controller that manages radiation emission according to commands received over a wireless or wired communication channel.

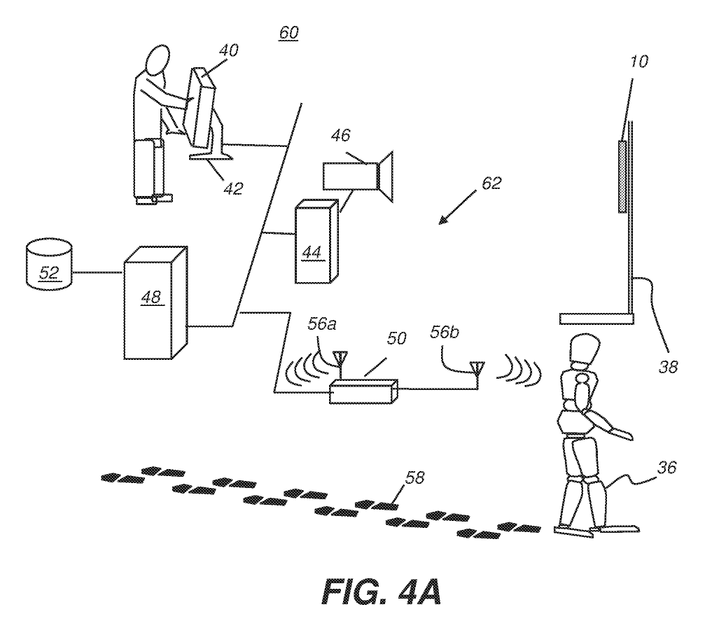

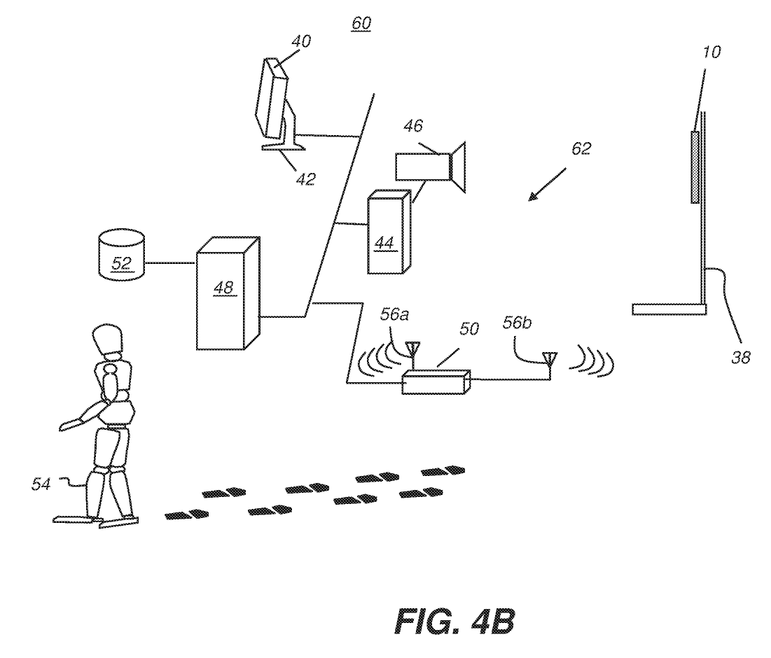

In the FIG. 3 arrangement, a host processor 48, such as a computer or dedicated processor apparatus, may be coupled to electronic memory 52 and coupled over a network to a wireless router 50, such as a WiFi router, that, acting as a transceiver for wireless RF (radio frequency) signals, such as signals in the 2.4 GHz range, may initialize wireless DR detector 10 to acquire radiographic image data during exposure of patient 36. Signal communication between router 50, host processor 48, control console 40, generator 44, and other devices may be provided by a wired or wireless network connection. The host processor 48, in signal communication with the wireless router 50, may be programmed to determine position or motion of objects or humans proximate the router antennae (FIG. 4A) according to the received wireless signals and, in response, to provide an output signal to activate the detector in preparation for image acquisition. In addition, the host processor 48 may be programmed to provide an output signal to activate the radiographic energy source in preparation for x-ray emission.

An embodiment of the present disclosure addresses the need for automatic power-up of wireless DR detector 10 in an x-ray imaging facility by detecting position of an animate or inanimate body, and changes in position indicating body motion utilizing the same wireless signals and signal handling mechanisms that are used by communication router 50. Signals emitted from router 50 and reflected from a body, such as a human body, are used in order to detect changes in position and related motion of any of patients or staff, and equipment motion between positions of interest within x-ray imaging site 60. In response to determining that positions of human practitioners, patients, or equipment indicate that an x-ray imaging exposure is anticipated, the host processor may initiate a signal to activate imaging system devices, as disclosed herein.

The schematic diagram of FIG. 4A illustrates tracking of a position and motion of a patient 36 or, additionally, of practitioner position and motion, using transmitted signals from wireless router 50. Motion in FIG. 4A, and following figures, is represented schematically by footprint tracings 58. Router 50 and processor 48 form a detection apparatus 62 for activation and deactivation of DR detector 10 or for activation and deactivation of x-ray source 46 and generator 44 to initiate and terminate radiographic imaging.

In order to use triangulation for position and motion sensing, router 50 of detection apparatus 62 includes two transceiver antennae 56a, 56b, sufficiently spaced apart to allow accurate position detection. When radio-frequency energy, or signals, is emitted by one or more of the antennae 56a, 56b, it is reflected back to the antennae 56a, 56b, from a moving person or other moving body or equipment. The emitted signal frequencies may be fixed, variable, the same, different, or a combination thereof, as between the set of antennae, which in one embodiment, exemplified herein, comprise two antennae. The frequency, or wavelength, of the received reflected electromagnetic (EM) wave or radio frequency (RF) energy is detectably altered by the reflection. This detectable change is caused by the familiar Doppler effect. For example, with patient 36 moving from left to right, as in the example of FIG. 4A, the sequence of emitted signals reflected back to router 50 may be used by processor 48 to calculate location, motion, direction of motion, and speed. Signals obtained from antennae 56a, 56b, through triangulation, may indicate the position of the moving body, providing sufficient information to detect normal activity, such as to ascertain whether or not a patient 36 is in position for imaging against bucky 38 or in some other suitable imaging position, for example. By way of further example, FIG. 4B shows detected practitioner 54 position and movement in an opposite direction compared to patient 36 in FIG. 4A. Using a coordinate map, such as an xy cartesian coordinate map, of the x-ray site 60 stored in memory 52, the processor 48 may determine, according to preprogrammed coordinate areas, that the position of one body, or a combination of two or more body locations, indicates that an x-ray exposure is about to take place at the x-ray site 60. Preprogrammed xy coordinates may be stored in memory 52 designating the floor area proximate the detector 10 that is mounted in wall stand bucky 38, which floor area is occupied by patient 36, as shown in FIG. 3. Additional xy coordinates may be stored in memory 52 to designate the floor area proximate the control console 40, which floor area is occupied by practitioner or technician 54, as shown in FIG. 3. In one embodiment, when movement or position of two bodies is determined by processor 48 to be proximate to, or to occupy, the stored xy coordinate areas, the processor 48 may be programmed to automatically activate, via wired or wireless signals, one or more x-ray imaging system components, as described herein. In one embodiment, when movement or position of one body is determined by processor 48 to be proximate to, or to occupy, a preprogrammed xy coordinate area, the processor 48 may be programmed to automatically activate, via wired or wireless signals, one or more x-ray imaging system components as described herein. In one embodiment, when movement or position of one body is determined by processor 48 to be proximate to, or to occupy, a preprogrammed xy coordinate area, and a second body is determined by the processor 48 to be moving closer to a preprogrammed xy coordinate area via triangulation computation, the processor 48 may be programmed to automatically activate, via wired or wireless signals, one or more x-ray imaging system components as described herein.

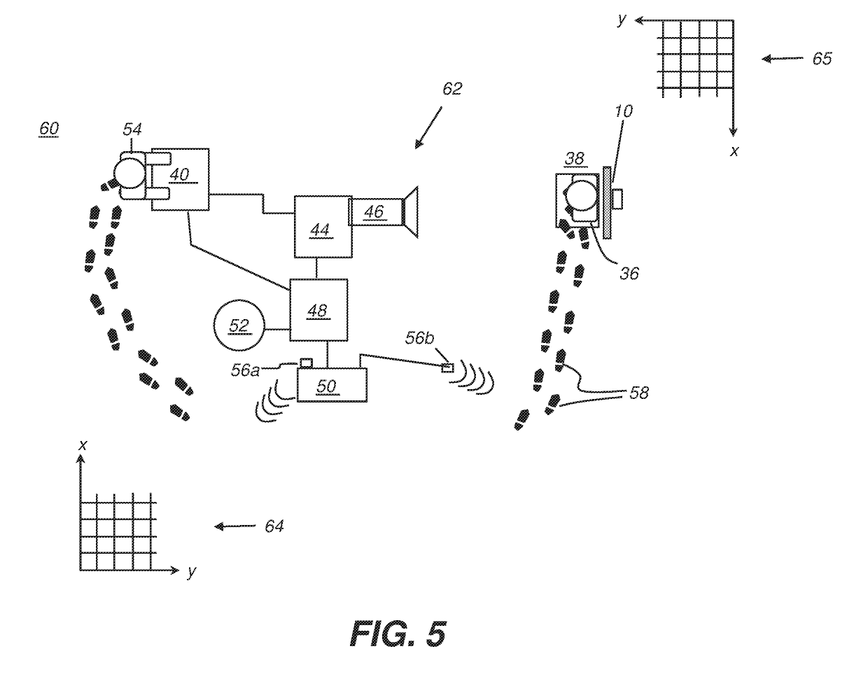

The schematic diagram of FIG. 5 is a top view of x-ray site 60 illustrating an exemplary detection of both practitioner 54 and patient 36 movement into position for x-ray radiographic imaging, as described above, and as also schematically shown by footstep tracings 58. Opposite corners of an exemplary xy coordinate system 64, 65, are illustrated in FIG. 5 and, in practice, may be extended to cover the entire area of x-ray site 60 that is within a detection area of router 50 antennae 56a, 56b. Router 50 signals emitted from antennae 56a, 56b may be reflected from nearby bodies and/or equipment back to the antennae to be processed at processor 48. This comparison of emitted signals and received reflected signals allows processor 48 to compute an xy coordinate location of practitioner 54, for example, and to determine whether practitioner 54 is within, or proximate to, a preselected and logically specified xy coordinate area near console 40, and to determine whether patient 36 is within, or proximate to, a preselected and logically specified xy coordinate area near detector 10, or otherwise within a preprogrammed suitable distance of these coordinate areas. Positional detection, or calculation, of one or both practitioner 54 and patient 36 within or proximate to the preprogrammed coordinate areas triggers an automatic programmed activation signal initiated at processor 48 and communicated to wireless router 50 to transmit signals to the digital DR detector to activate the digital DR detector 10 to a ready state for radiographic imaging. Generator 44 and X-ray source 46 may also be similarly activated to a ready state using such wireless signals from router 50, as described herein.

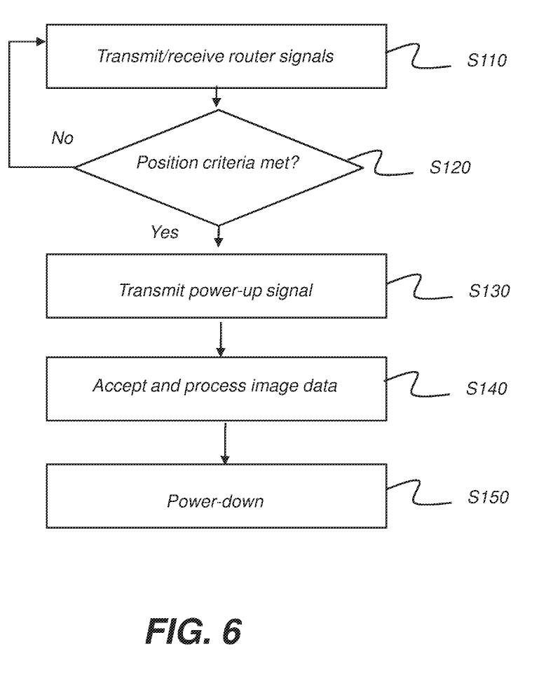

FIG. 6 is a logic flow diagram for power-up and imaging processing that may be executed using the x-ray site apparatus shown in FIG. 5. In a transmit/receive step S110, router 50 initiates RF signal emission and reception, using one or more antennae. The received signals are processed to detect movement of either or both the patient 36 and practitioner 54, and, alternatively, of one or more needed apparatuses, into preprogrammed xy coordinate areas for radiographic imaging. In a criteria test step S120, programmed processor 48 logic may access electronic memory 52 to obtain one or more preselected xy coordinate sets that identify areas within the x-ray site 60, and to check that determined locations of either or both practitioner 54 and patient 36 indicates that either or both are within or proximate to the preselected areas. This involves detecting either or both practitioner 54 and patient 36 position as computed by processor 48 using received RF signals at antennae 56a, 56b, communicated to the host processor 48 via router 50. Other criteria may include equipment positioning and configuration, for example. Once the criteria are satisfied, a power up program step S130 at processor 48 automatically initiates a transmission of an initialization signal from router 50 to DR detector 10. This energizes DR detector 10 to perform any necessary initialization, clearing of buffers, refreshing of signals, or other activity needed in preparation for receiving and processing an x-ray exposure. With DR detector 10 in a ready state, possibly after a slight timing delay, an imaging step S140 executes. Entry to a ready or an imaging state can be verified or assisted using a manual process, such as caused by the x-ray technician or practitioner pressing a prep/expose switch. Imaging step S140 acquires image data at DR detector 10 which may be transmitted to host processor 48. According to an embodiment of the present disclosure, an audible tone, on-screen message, or other indicator can be provided, actuated or highlighted in order to indicate that the DR detector has been activated to the Ready state for image acquisition. After the x-ray image has been received, a power down step S150 may execute, causing router 50 to transmit a power down signal to DR detector 10.

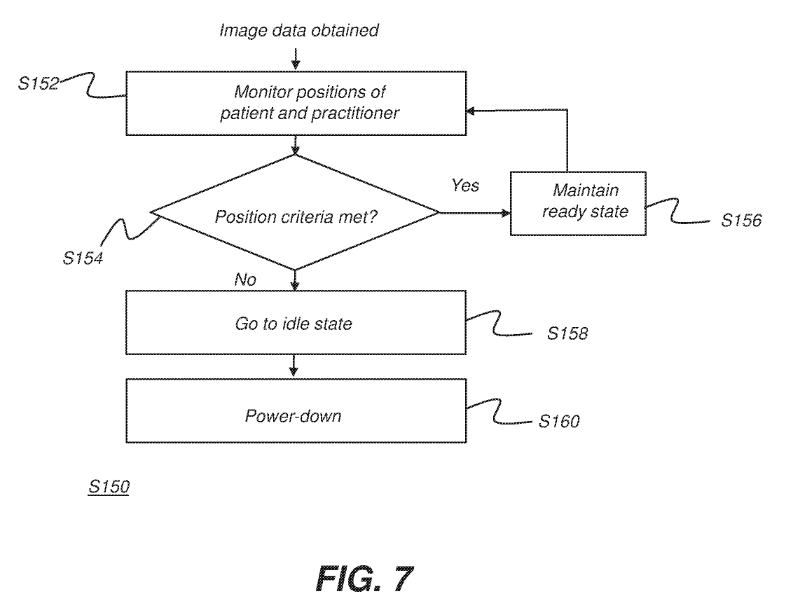

Power down step S150 execution may be based upon detection of particular conditions at the x-ray site, as shown in the flow diagram of FIG. 7. For example, in a monitoring step S152, host processor 48 may process signals from router 50 to determine when the patient 36 has moved away from the wall stand 38 or from some other position. Similarly, movement of the practitioner or technician 54 away from the control console 40 can be detected. In a criteria test step S154, one or both of these satisfied conditions may be programmed to initiate power-down or to maintain a ready state in step S156. Detected movement of only the practitioner 54 out of the area near the control console 40, for example, while the patient 36 remains in the designated coordinate area near the detector 10 and wall stand 38, may indicate that the practitioner is involved with re-positioning the patient 36, and so does not require a programmed power-down. Such detected movement sequences may be addressed by suitable program steps handled by host processor 48. A combination of detected movements may also be used to confirm that patient imaging has been completed. Where a detected movement pattern indicates the end of the imaging session, power-down can continue through any number of stages. As shown in FIG. 7, the host processor 48 may instruct the DR detector 10 to transition to an idle state in an optional idle state step S158. An idle state can be an intermediate state between full readiness and power down. After a suitable time interval in the idle state and after monitoring detected position and movement information, a power down step S160 may be executed to reduce power consumption of the DR detector until the next imaging session.

It can be appreciated that there can be any number of variations and refinements to the basic process described with reference to FIGS. 6 and 7, including steps to confirm DR detector initialization, timeout processes, and periodic refreshing of the DR detector circuitry until the x-ray exposure energy is received. Criteria test step S120 in FIG. 6 maybe used to automatically trigger DR detector 10 power-up before the patient 36 is actually within a preselected xy coordinate position, or may be delayed until practitioner 54 has stopped moving, for example. DR detector 10 can have a number of intermediate states between initial power-up and full readiness for imaging, and may signal readiness through any number of signals or audio or visual indicators, for example. Site specific programming can be used to set different variables, including timeout intervals or delay intervals.

Router 50 may have more than two antennae, positioned at various places around the x-ray site 60. In addition to embodiments using wall-mounted equipment, systems that mount the DR detector 10 on a C arm or beneath a patient bed or platform may also use the automated power-up features described herein. Systems using ceiling-mount or mobile x-ray sources may also use these features.

According to an embodiment of the present disclosure, host processor 48 software at x-ray site 60 may be programmed to detect various conditions for automatic power-up of the DR detector. Power-up criteria may be adapted for the environment, particular equipment layout, and workflow practices of a given x-ray facility. DR detector 10 may be adapted to work with any of a number of different types of x-ray equipment and to conform to specific requirements for each x-ray site 60.

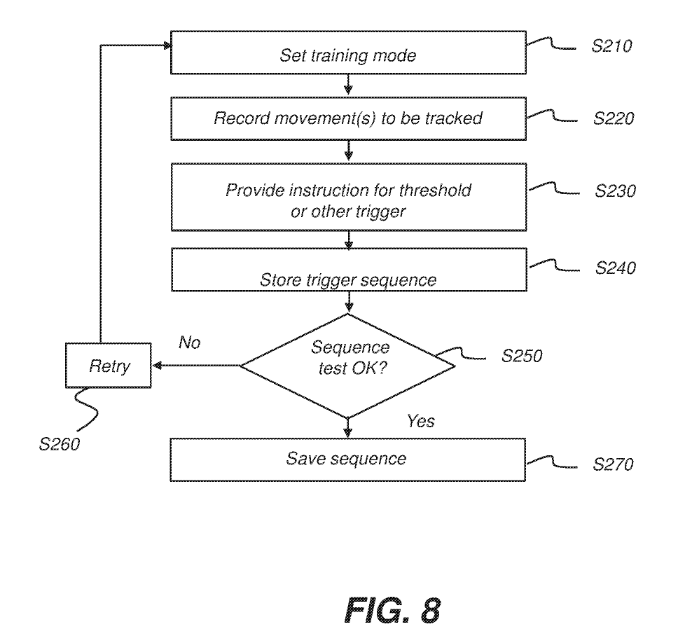

The exemplary logic flow diagram of FIG. 8 shows a sequence that may be used for site-specific customization setup and programming or “training” of the site 60 apparatuses for wireless DR detector power-up function. In one embodiment, in a set training mode step S210, the operator enters an instruction that invokes the training function. Initialization of this function can include “learning” the locations of components in the x-ray room itself, so that any change in position, such as that caused by moving persons or by shifting apparatus location, can be detected at the host processor 48 and used to trigger the DR detector 10. Training can also condition the processor 48 logic to “learn” the stationary positions of equipment within the x-ray room. The patient/practitioner movement and positioning shown with reference to FIG. 5 are detected, recorded, and used for triggering power-up. It should be noted that a similar approach can be used to detect and respond to changes in equipment movement and placement related to imaging procedure.

According to an alternate embodiment of the present disclosure, detected movement patterns can cause processor 48 to generate and display a prompt to the practitioner, requesting verification of detector 10 activation, for example. This can be useful where sensed movement patterns are ambiguous, but may indicate that power-up is desirable. In such a case, a command entry from control console 40 (FIG. 3) may be used to generate the activation signal.

Various lockout functions can also be programmed as part of logic training for preventing exposure at a site unless required conditions are met. Motion detection by detection apparatus 62, for example, may determine that patient motion is excessive. Detection of this condition may cause processor 48 to disable exposure until motion is at acceptable levels or until an operator override is entered. As another example, the patient position may not meet programmed requirements, causing the processor 48 to block exposure unless the condition is corrected or an operator override is entered.

In one embodiment, with reference to FIG. 8, in a record step S220, the movement patterns of interest may be enacted, such as by having two participants move into the area of site 10 and take standard positions for patient and practitioner as shown in FIG. 5, for example. In a threshold setting step S230, user instructions relating to interpretation of the movement patterns are entered, such as by storing xy coordinate data pertaining to the imaging-start positions for patient and practitioner. Acceptable proximity ranges may be entered and stored at this time as well as ranges for movement toward one or more of the preselected xy coordinate areas. For example, movement toward the bucky, followed by stopped movement at the bucky, can be programmed to be interpreted as a positioning of the patient. Similarly, movement toward the control console, followed by stopped movement for a suitable interval at the console, may indicate positioning of the practitioner or technician for imaging. A store step S240 then temporarily stores the motion and position sequence for later automatic triggering of a power-up activation based on steps S220 and S230. A test step S250 can be used to check for the desired power-up behavior, re-enacting movement of patient and practitioner and determining if power-up response is appropriately carried out. A retry step S260 enables correction or adjustment of movement detection patterns or of threshold trigger settings. When the sequence tests successfully, a save step S270 then saves the totality of parameters for use within the x-ray imaging site 60.

The training function described with reference to FIG. 8 may also be repeated one or more times during normal use of the equipment. Repeating this training sequence during imaging with actual practitioners and patients can help to improve overall detection accuracy and help to reduce the likelihood of false detections, for example. According to an alternate embodiment, a technician or practitioner can be trained to follow specific movement patterns in order to initiate DR detector power-up. For example, the detection apparatus 62 can be trained to detect and interpret one or more positions of the practitioner to indicate that power-up should be initiated.

In one embodiment, adding one or more additional antennae to existing wireless communication apparatus allows detection of people and other bodies in motion. “Detector readiness” may refer to any of a number of device states through which the DR detector advances from a powered down or inactive state to one or more successive initialization states that ready the DR detector for image generation and processing. In a retrofit installation, for example, where the DR detector replaces a film cassette or computed radiography (CR) detector plate originally provided with a radiographic imaging system, there may be no built-in mechanism for automatic power-up and preparation of the DR detector prior to imaging. Requisite steps for achieving readiness can include refreshing of memory contents, clearing of accumulated image signal content from image data registers, and other steps needed prior to accepting radiation and generating digital data indicative of radiation at particular points along the DR detector.

The method of the present disclosure can also provide a computer storage product having at least one computer storage medium having instructions stored therein causing one or more computers to perform the described calculations and provide signals needed for initialization and imaging.

Consistent with one embodiment, the present invention utilizes a computer program with stored instructions that control system functions for sensor data acquisition and processing. As can be appreciated by those skilled in the data processing arts, a computer program of an embodiment of the present invention can be utilized by a suitable, general-purpose computer system, such as a personal computer or workstation that acts as an image processor, when provided with a suitable software program so that the processor operates to acquire, process, transmit, store, and display data as described herein. Many other types of computer systems architectures can be used to execute the computer program of the present invention, including an arrangement of networked processors, for example.

The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present invention may make use of various data manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the sensor and signal processing arts. Additional aspects of such algorithms and systems, and hardware and/or software for producing and otherwise processing the acquired data or co-operating with the computer program product of the present invention, are not specifically shown or described herein and may be selected from such algorithms, systems, hardware, components and elements known in the art.

The invention has been described in detail, and may have been described with particular reference to a suitable or presently preferred embodiment, but it will be understood that variations and modifications can be effected within the spirit and scope of the invention. In addition, while a feature(s) of the invention can have been disclosed with respect to only one of several implementations/embodiments, such feature can be combined with one or more other features of other implementations/embodiments as can be desired and/or advantageous for any given or identifiable function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A radiographic imaging system comprising:
a radiographic energy source;
a digital radiographic detector including a wireless transceiver;
a wireless router disposed to transmit and to receive electromagnetic signals from a pair of antennae spaced apart from each other; and
a host processor in signal communication with the wireless router, the host processor programmed to determine position or motion proximate the first and second antennae according to the received signals and, in response to the determined position or motion, to provide an output signal to activate the detector in preparation for image acquisition.

2. The system of claim 1, wherein the host processor is further in signal communication with the radiographic energy source to provide an output signal to activate the radiographic energy source in preparation for x-ray emission.

3. The system of claim 2, wherein the host processor is programmed to determine whether a position of a body is within or proximate to a patient imaging area.

4. The system of claim 3, wherein the host processor is programmed to determine whether a position of a body is within or proximate to a technician control area.

5. The system of claim 1, wherein the host processor is programmed to determine position or motion with reference to a stored two dimensional cartesian coordinate map.

6. The system of claim 1, wherein the pair of antennae is configured to receive reflected electromagnetic waves, and wherein the host processor determines position or motion based on the reflected electromagnetic waves.

7. A radiographic imaging facility comprising:
a radiographic detector including a first wireless transceiver;
a host computer system; and
a second wireless transceiver including first and second antennae spaced apart from each other, the second wireless transceiver in signal communication with the host computer system and with the radiographic detector, the first and second antennae each configured to transmit radio waves into the facility,
wherein the host computer system is configured to detect human body movement within the facility according to detected reflected radio waves at the first and second antennae, and to transmit an activation signal to the radiographic detector when the detected human body movement satisfies predetermined criteria.

8. The facility of claim 7, wherein the host computer system is in signal communication with a radiographic energy source to transmit an activation signal to the radiographic energy source when the detected human body movement satisfies the predetermined criteria.

9. The facility of claim 7, wherein the predetermined criteria includes determining that a position of a body is within or proximate to a patient imaging area.

10. The facility of claim 9, wherein the predetermined criteria further includes determining that a position of a practitioner is within or proximate to a control console area.

11. The facility of claim 7, wherein the host computer system is configured to determine human body movement with reference to a stored two dimensional cartesian coordinate map of the radiographic imaging facility.

12. The facility of claim 7, wherein the first and second antennae are positioned within an x-ray imaging room.

13. A method for radiographic imaging comprising:
transmitting electromagnetic signals from a set of antennae;
receiving reflections of the transmitted electromagnetic signals and, in response thereto, detecting a position of a body;
transmitting a power-up signal to a wireless digital radiography detector in response to the step of detecting the position of the body; and
acquiring a radiographic image of at least a portion of a patient from the detector.

14. The method of claim 13, wherein the step of detecting the position of the body comprises detecting a position of the patient.

15. The method of claim 13, further comprising transmitting a power-down signal to the detector after the step of acquiring the radiographic image.

16. The method of claim 13, further comprising detecting a sequence of positions of the body before the step of transmitting the power-up signal.

17. The method of claim 13, further comprising training a processor to detect the position of the body.

18. The method of claim 13, wherein the body is a patient or an x-ray technician.
